(19) United States
(12) Patent Application Publication
(10) Pub. No.: US 2024/0381950 A1
(43) Pub. Date: Nov. 21, 2024

(54) MOISTURE ABSORBENT BRA

(71) Applicant: MILK LABEL PTY LTD, Indooroopilly, QLD (AU)

(72) Inventors: Stephanie MCMURDY, Milton, QLD (AU); D’ABADIE Jessica, Milton, QLD (AU)

(21) Appl. No.: 18/686,849

(22) PCT Filed: Aug. 25, 2022
(86) PCT No.: PCT/AU2022/051028

§ 371 Date: Feb. 26, 2024

(30) Foreign Application Priority Data
Aug. 25, 2021 (AU) 2021107364

(51) Int. Cl. A41C 3/04; A41C 3/00

(52) U.S. Cl. CPC A41C 3/04; A41C 3/0035

(57) ABSTRACT

A washable moisture absorbent bra for lactating persons is provided that is simple to manufacture, may have improved absorbency and moisture barrier properties, may be more comfortable and enable more freedom of movement, and is environmentally friendly. The bra comprises: first and second cup portions defined by inner and outer layers, the inner layer positioned against the breast of the lactating person when used; and first and second inserts positioned intermediate the inner and outer layers in the first and second cup portions. The inserts comprise a moisture absorbent inner portion, and an outer moisture barrier bonded to the absorbent inner portion. The first and second inserts are provided in central parts of the first and second cup portions such that an insert-free portion is defined between an edge of the first and second inserts and an edge of the first and second cup portions.

### TECHNICAL FIELD

The present invention relates to bras. In particular, the present invention relates to moisture absorbing bras.

### BACKGROUND ART

A problem for lactating persons has long been that breast milk tends to leak and stain clothing, which is clearly undesirable.

Several attempts have been made to absorb breast milk in such circumstances, including with disposable absorbent pads (nursing pads). In particular, absorbent pads are positioned in such case on an inside of the bra to absorb any such leakage.

Several problems exist with these disposable absorbent pads. In particular, the pads may move or become folded, resulting in reduced function and discomfort. Furthermore, the pads do not have high absorbency, which may also result in leakage outside the pads. Furthermore, such disposable pads are inconvenient, as replacement pads must be carried, and are not environmentally friendly, as they are discarded.

Certain absorbent bras exist that include either removable or fixed absorbent structures to absorb leakage from the breasts. One problem with such absorbent bras is that they may not provide good leak resistance and may not have good absorbency characteristics.

Another problem with such bras, particularly those with fixed absorbent structures, is that they may lose their leak resistance abilities over time, particularly when washed. This may particularly be the case for materials which are chemically treated (e.g. using a hydrophobic treatment) to provide leak resistance.

Yet another problem with such bras is that they generally have a quite rigid structure, and may be uncomfortable, and not able to adapt well with breast size changes. Furthermore, this structure may restrict freedom of movement.

As such, there is clearly a need for improved moisture absorbent bras.

It will be clearly understood that, if a prior art publication is referred to herein, this reference does not constitute an admission that the publication forms part of the common general knowledge in the art in Australia or in any other country.

### SUMMARY OF INVENTION

The present invention relates to moisture absorbent bras, which may at least partially overcome at least one of the abovementioned disadvantages or provide the consumer with a useful or commercial choice.

With the foregoing in view, the present invention in one form, resides broadly in a washable moisture absorbent bra for lactating persons comprising:
first and second cup portions defined by inner and outer layers, the inner layer positioned against the breast of the lactating person when used;
first and second inserts positioned intermediate the inner and outer layers in the first and second cup portions, the inserts comprising a moisture absorbent inner portion, and an outer moisture barrier bonded to the moisture absorbent inner portion,
wherein the first and second inserts are provided in central parts of the first and second cup portions such that an insert-free portion is defined between an edge of the first and second inserts and an edge of the first and second cup portions.

Advantageously, the bra is simple to manufacture, as it can be cut and sewn without shaping or molding the cup, as the inserts need not conform in shape to the entire cups. Furthermore, the moisture absorbent bra may have improved absorbency and moisture barrier properties, particularly when compared with corresponding molded bras. Yet further again, the bra may be more comfortable and enable more freedom of movement, particularly when compared with corresponding molded bras. Being washable (and thus reusable) the bra is more environmentally friendly than single use breast pads.

Preferably, the insert-free portions of the first and second cups each comprise a peripheral edge around the first and second inserts. The insert-free portions may include an insert-free portion between the inserts and a seam associated with an edge of the cup portions.

The inner and outer layers may be formed of any suitable material. In a preferred embodiment, the inner and/or outer layers are formed of wool. The inner and/or outer layers may be formed of merino wool. Alternatively, the inner and/or outer layers may be formed of bamboo, cotton, polyester, hemp, rayon, modal, silk, Tencel/Lyocell, wool, recycled fabrics, or any combination thereof.

The inner and/or outer layers may be knitted. The inner and/or outer layers may be formed of Jersey knit fabric. The inner and/or outer layers may be stretch fabric.

The first and second inserts may each be attached to the inner layer. The first and second inserts may each be attached to the inner layer and not the outer layer.

The first and second inserts may each be attached to the inner layer at or adjacent to a peripheral edge of the first and second inserts. The first and second inserts may be stitched to the inner layer.

The first and second inserts may comprise moisture absorbent pads. The moisture absorbent pads may be less than about 3 mm thick. The moisture absorbent pads may be less than about 2 mm thick. The moisture absorbent pads may be about 1.5 mm thick.

The moisture absorbent pads may be between about 300 GSM and 600 GSM. The moisture absorbent pads may be about 425 GSM.

The moisture absorbent inner portion may be made of any suitable material. In a preferred embodiment, the moisture absorbent inner portion is formed of polyester. Preferably, the moisture absorbent inner portion is knitted or woven.

The moisture barrier may be made of any suitable material. In a preferred embodiment, the moisture barrier may comprise polyurethane film.

The moisture barrier may be thermally bonded to the moisture absorbent inner portion.

The inserts may be substantially uniform in cross-section.

The inserts may be substantially planar, when not used.

The inserts may be at least partially cup-shaped. The inserts may be formed of a planar material, including a dart, to create the cup-shape.

The inner and outer layers may extend continuously into and define straps of the bra, for supporting the bra over the shoulders of the wearer. Similarly, the inner and outer layers may extend continuously from respective sides of the cups to a rear of the bra.

The bra may include an elastic band that extends around a torso of the wearer.

The bra may comprise a nursing bra. The nursing bra may include one or more clamps or fasteners, to enable the cups to open, exposing the breasts. The cups may fold downwards.

Preferably, the bra is a wireless bra.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 illustrates a front view of a moisture absorbent bra, according to an embodiment of the present invention.

FIG. 2 illustrates a rear view of the moisture absorbent bra of FIG. 1, according to an embodiment of the present invention.

FIG. 3 illustrates a cross-sectional exploded view of a portion of the bra of FIG. 1 illustrating an absorbent insert thereof, according to an embodiment of the present invention.

FIG. 4 illustrates an internal (forward facing) view of a front of the bra FIG. 1, according to an embodiment of the present invention.

FIG. 5 illustrates an internal (forward facing) view of a front of a bra, according to an alternative embodiment of the present invention.

FIG. 6 illustrates an internal (forward facing) view of a front of a bra, according to an alternative embodiment of the present invention.

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

### DESCRIPTION OF EMBODIMENTS

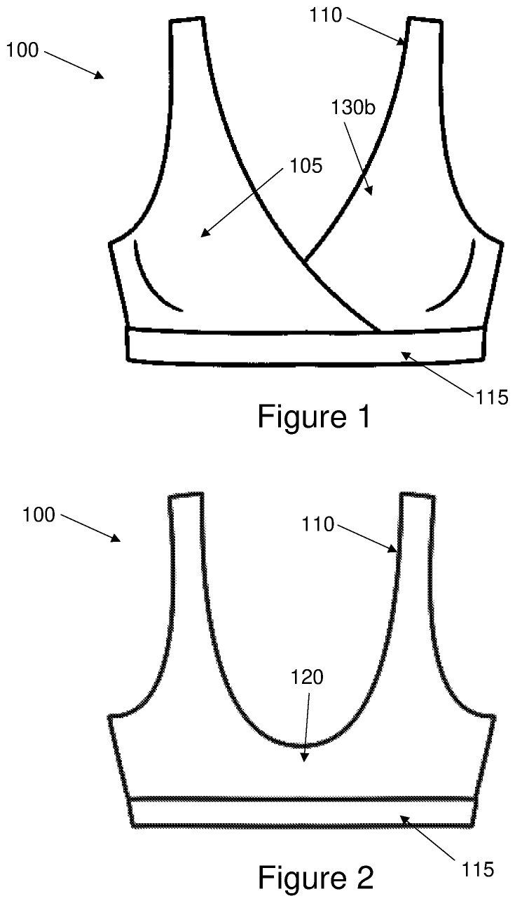

FIG. 1 illustrates a front view of a moisture absorbent bra 100, and FIG. 2 illustrates a rear view of the moisture absorbent bra 100, according to an embodiment of the present invention.

Advantageously, the bra 100 is absorbent, and configured to absorb leakage from the breasts of lactating persons. The bra 100 is washable, and therefore more environmentally friendly than single use breast pads. Furthermore, the bra may be more comfortable than other absorbent bras, or bras used with absorbent breast pads, and provide greater freedom of movement.

The bra 100 comprises first and second cup portions 105, which conform to the shape of breasts of a wearer, straps 110, which extend from the cup portions 105 and over a shoulder of the wearer. The straps 110 extend down at a back portion to an elastic band 115, which elastic band 115 extends around a torso of the wearer. The elastic band 115 is also joined to a lower edge of the cup portions 105 at the front.

A rear panel 120 extends from sides of the first and second cup portions 105, and around to a rear of the bra 100, above the elastic band 115. The straps 110 extend continuously into the rear panel 120, and continuously from upper parts of the cup portions 105. As the straps 110, rear panel 120 and cup portions 105 extend continuously into each other, no well-defined boundaries, such as seams, exist between these portions. In other embodiments, the bra 100 may be separable at the rear by a clip, e.g. a hook and eye clip or closure.

The skilled addressee will readily appreciate that a variety of other designs may, however, be used without deviating from the scope of the invention.

The bra 100 includes absorbent inserts 125 in cup portions 105, configured to absorb leakage from the breasts when worn. The absorbent inserts 125 are sandwiched between inner and outer layers 130a, 130b of the bra 100, as outlined below, and are therefore not visible in FIG. 1. The stitching is not visible from an outside when worn, and as such, the absorbent inserts 125 are concealed within the bra 100.

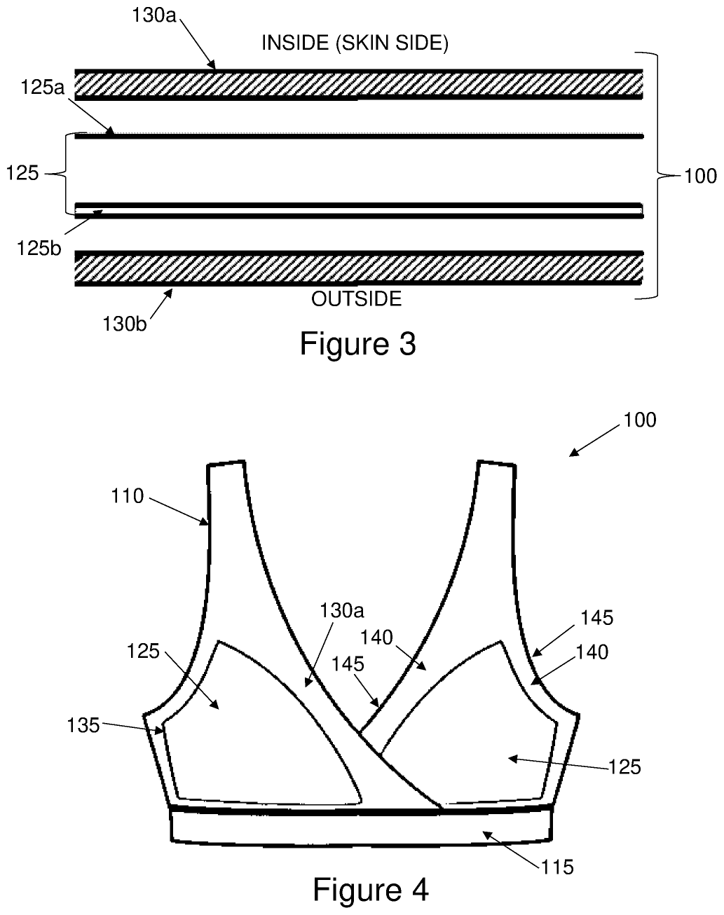

FIG. 3 illustrates a cross-sectional exploded view of a portion of the bra 100 including an absorbent insert 125, according to an embodiment of the present invention.

The absorbent insert 125 comprises an absorbent layer 125a, on which a waterproof layer 125b is bonded, and on an outside surface (i.e. away from the breasts) thereof.

The absorbent insert 125 is sandwiched between inner and outer 130a, 130b layers, which advantageously comprise Jersey knit fabric of merino wool, but the skilled addressee will readily appreciate that other materials, such as bamboo, cotton, polyester, hemp, rayon, modal, silk, Tencel/Lyocell, wool, recycled fabrics, or any combination thereof.

The absorbent insert 125 is stitched around its periphery to the inner layer 130a only, and thus not to the outer layer 130b. The insert is not bonded to the inner or outer layers 130a, 130b.

FIG. 4 illustrates an internal (forward facing) view of a front of the bra 100, according to an embodiment of the present invention.

The stitching 135 extends around an edge of the absorbent insert 125, to stitch the absorbent insert 125 to the inner layer 130a, and therefore shows an outline of absorbent insert 125 in FIG. 4. The stitching is advantageously a row of zigzag or flatlock stitching around the outer edge of the insert.

The first and second inserts are provided in central parts of the first and second cup portions 105, such that an insert-free portion 140 is defined between an edge of the first and second inserts and an edge of the first and second cup portions.

The insert-free portion 140 defines a peripheral edge (flange) around the first and second absorbent inserts 125, such that the absorbent inserts 125 do not extend to edges of the cup portions 105, and do not extend to seams 145 associated with the edges of the cup portions 105. This enables the cup portions 105 to conform to the shape of the breast through stretching of the insert-free portion 140 more than the portion of the cup 105 including the absorbent insert 125.

Such configuration also provides greater freedom of movement of the wearer, and enables the bra 100 to be worn comfortably when exercising, performing yoga, stretching, or the like. The peripheral edge being stretch fabric allows for expansion and movement in the cup portions 105 relative to the seams and non-cup portions of the bra.

Furthermore, such configuration provides greater size flexibility, and thus allows for greater size diversity and fluctuating breast sizes within each size bracket, which is particularly useful as the breasts will generally change in size while lactating. This is in clear contrast to molded bras, where cup sizes are generally fixed and do not adapt to changing or diverse breast sizes.

Yet further again, as the absorbent inserts 125 are not bonded, the structure can move and therefore allows for any leakage to flow freely through the layers of the bra 110 into the inserts 125, and also within the inserts 125.

While the above-described example bra 100 includes absorbent inserts 125 of a particular size and shape, the skilled addressee will readily appreciate that a wide variety of other shapes may be used.

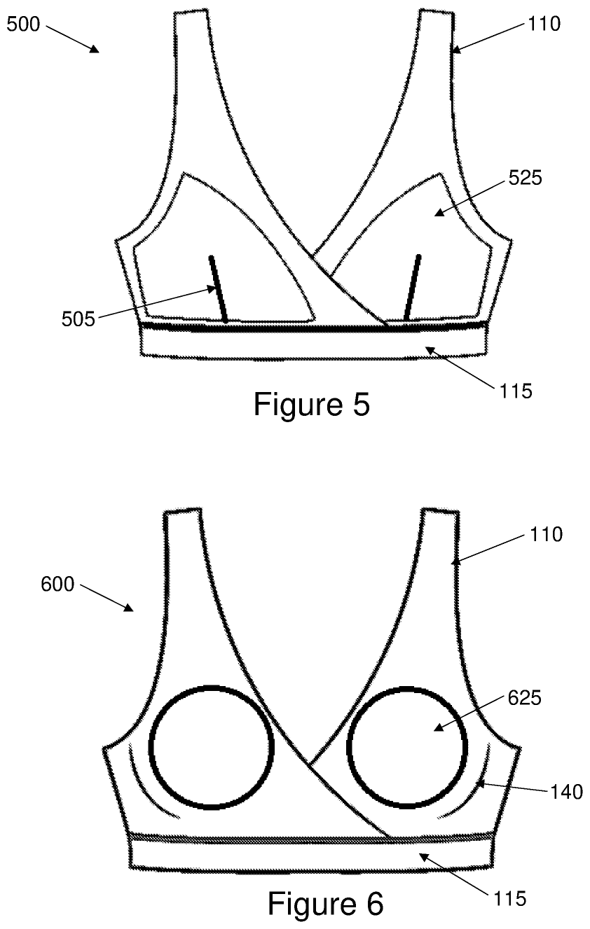

FIG. 5 illustrates an internal (forward facing) view of a front of a bra 500, according to an alternative embodiment of the present invention.

The bra 500 is similar to the bra 100, but wherein the absorbent insert 525 is three-dimensional in shape (rather than flat). In particular, a dart 505 is cut and sewn into the absorbent insert 525, such that the absorbent insert 525 becomes cup-shaped, and thereby more accurately conforms to the shape of the wearers breast.

The size of the dart 505, and shape thereof, may vary depending on the size of the bra 500, as will be appreciated by the skilled addressee.

FIG. 6 illustrates an internal (forward facing) view of a front of a bra 600, according to an alternative embodiment of the present invention.

The bra 600 is similar to the bra 100, but wherein the absorbent insert 625 is circular in shape. Such configuration provides larger non-insert 140 areas at sides of the bra 100, which may be beneficial from a flexibility or freedom of movement perspective.

Now turning back to composition of the absorbent inserts 125 (which may have a similar or identical composition/layering to absorbent inserts 525, 625, the absorbent layer 125a comprises knitted or woven polyester. The polyester is knitted or woven to provide a three-dimensional structure that is suited for moisture retention, and which facilitates distribution of moisture laterally across the absorbent inserts 125. In contrast to super absorbent polymer (SAP) granules, for example, the knitted or woven structure is more resilient, and holds its shape during washing.

The waterproof layer 125b comprises polyurethane film that is thermally bonded to the absorbent layer 125a. By thermally bonding (melt-bonding) the polyurethane film, toxic solvents and the like can be avoided, resulting in a product that is non-toxic and thus safe to wear, which is particularly important around infants and babies.

The absorbent inserts 125, 525, 625 may be about 1.5 mm thick, and about 425 grams per square meter (GSM).

Advantageously, the bra described above is simple and cost-effective to manufacture as it can be cut and sewn without shaping or molding the cup.

As the bra is cut and sewn without shaping or molding, a wider range of fabrics and materials may be used. In addition to enabling merino wool fabrics to be used, which are not able to be moulded, a wider range of waterproof materials may be used, which may otherwise not be able to be used with the moulding process. The molding process used in other bras may also reduce the waterproof/resistant characteristics of materials, as the heating process may fade or melt away part of the water resistant materials.

As the inserts are not attached to any exterior seams, and an insert-free portion extends around the inserts, bra may be more comfortable and enable more freedom of movement, particularly when compared with corresponding molded bras, even when the insert is fairly rigid.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A washable moisture absorbent bra for lactating persons comprising:
first and second cup portions defined by inner and outer layers, the inner layer positioned against the breast of the lactating person when used;
first and second inserts positioned intermediate the inner and outer layers in the first and second cup portions, the inserts comprising a moisture absorbent inner portion, and an outer moisture barrier bonded to the absorbent inner portion,
wherein the first and second inserts are provided in central parts of the first and second cup portions such that an insert-free portion is defined between an edge of the first and second inserts and an edge of the first and second cup portions.

2. The washable moisture absorbent bra of claim 1, wherein the insert-free portions of the first and second cups comprises a peripheral edge around the first and second inserts.

3. The washable moisture absorbent bra of claim 1, wherein the insert-free portions include an insert-free portion between the inserts and a seam associated with an edge of the cup portions.

4. The washable moisture absorbent bra of claim 1, wherein at least one of the inner and outer layers are knitted.

5. The washable moisture absorbent bra of claim 1, wherein at least one of the inner and outer layers comprise stretch fabric.

6. The washable moisture absorbent bra of claim 1, wherein at least one of the inner and outer layers are formed of merino wool.

7. The washable moisture absorbent bra of claim 1, wherein the first and second inserts are each be attached to the inner layer.

8. The washable moisture absorbent bra of claim 1, wherein the first and second inserts are each attached to the inner layer and not the outer layer.

9. The washable moisture absorbent bra of claim 1, wherein the first and second inserts are each attached to the inner layer at or adjacent to a peripheral edge of the first and second inserts.

10. The washable moisture absorbent bra of claim 9, wherein the first and second inserts are stitched to the inner layer.

11. The washable moisture absorbent bra of claim 1, wherein the first and second inserts comprise moisture absorbent pads.

12. The washable moisture absorbent bra of claim 11, wherein the moisture absorbent pads are less than about 3 mm thick.

13. The washable moisture absorbent bra of claim 11, wherein the moisture absorbent pads are between about 300 GSM and 600 GSM.

14. The washable moisture absorbent bra of claim 1, wherein the moisture barrier comprises polyurethane film.

15. The washable moisture absorbent bra of claim 1, wherein the inserts are substantially uniform in cross-section.

16. The washable moisture absorbent bra of claim 1, wherein the inserts are at least partially cup-shaped.

17. The washable moisture absorbent bra of claim 1, wherein the inner and outer layers extend continuously into and define straps of the bra, for supporting the bra over the shoulders of the wearer.

18. The washable moisture absorbent bra of claim 1, wherein the inner and outer layers extend continuously from respective sides of the cup portions to a rear of the bra.

19. The washable moisture absorbent bra of claim 1, comprising a nursing bra, the nursing bra including one or more clamps or fasteners, to enable the cup portions to open, exposing the breasts.

20. The washable moisture absorbent bra of claim 1, comprising a wireless bra.
